SEQUENCE LISTING (2 sequences)

SEQ ID NO: 1 - DNA, 513 bp, Homo sapiens
  CDS (1)..(513)
atgtctgtggccggtggggagattcgtggggacacggggggagaggacactgctgctcccggccggttcagcttcagcccggagcccacgctcgaggacatccgccgcctccatgctgagtttgctgcggaacgagactgggaacagttccatcagcctcggaatctcctcctggccttggttggggaagtgggggagctggcagaactctttcagtggaaaaccgatggggaacctggcccccaaggctggtcccccagggaacgggcagcccttcaagaggagcttagtgacgtcctcatctacctggtggcattagcagcccgctgccgtgtggatctgccgctagcagtgctctccaaaatggacatcaaccggcgacgctacccagcccatctggcccgcagctcttcccgcaagtatacagaattgccccatggggccatctctgaagaccaggctgtggggcctgcggacattccctgtgactccacaggccagacctcaacctag

SEQ ID NO: 2 - protein, 170 aa, Homo sapiens
MSVAGGEIRGDTGGEDTAAPGRFSFSPEPTLEDIRRLHAEFAAERDWEQFHQPRNLLLALVGEVGELAELFQWKTDGEPGPQGWSPRERAALQEELSDVLIYLVALAARCRVDLPLAVLSKMDINRRRYPAHLARSSSRKYTELPHGAISEDQAVGPADIPCDSTGQTST

(19) United States
(12) Patent Application Publication
(10) Pub. No.: US 2003/0157111 A1
(43) Pub. Date: Aug. 21, 2003

(54) 76A11 polypeptide and uses thereof

(76) Inventors: Ayelet Chajut, Ramat Hasharon (IL); Matat Levinson, Nes Zionna (IL); Rami Skaliter, Nes Zionna (IL)

Correspondence Address: John P. White, Cooper & Dunham LLP, 1185 Avenue of the Americas, New York, NY, 10036, US

(21) Appl. No.: 10/128,736

(22) Filed: Apr. 23, 2002

Related U.S. Application Data
(60) Provisional application No. 60/285,896, filed on Apr. 23, 2001.
(60) Provisional application No. 60/333,566, filed on Nov. 27, 2001.

(51) Int. Cl. C12Q 1/68; G01N 33/574; C07H 21/04; A61K 48/00; A61K 39/395; A61K 38/18; C07K 14/475

(52) U.S. Cl. 424/155.1; 424/93.21; 514/12; 435/6; 530/399; 536/23.5; 435/7.23

(57) ABSTRACT

This invention provides a method for treating a subject suffering from a disease, wherein the disease is characterized by aberrant cellular proliferation or aberrant cellular differentiation, comprising administering to the subject an antagonist of a polypeptide, wherein the polypeptide has an amino acid sequence as presented in SEQ ID NO:2. This invention also provides the use of a polynucleotide as a probe for identifying undifferentiated embryonic stem cells, wherein the polynucleotide comprises a nucleotide sequence as presented in SEQ ID NO:1. This invention further provides the use of the 76A11 polypeptide in vivo or ex vivo for expansion of adult somatic stem cells. This invention further provides the use of an antibody directed to an epitope presented by the polypeptide comprising the amino acid sequence as presented in SEQ ID NO:2 for detection of cancerous cells or of undifferentiated stem cells. This invention further provides the use of a polynucleotide comprising the nucleotide sequence as presented in SEQ ID NO:1 or fragments thereof for identifying cancerous cells. This invention further provides a method for identifying a chemical compound that modulates proliferation or differentiation of cells which comprises: (a) contacting a cell expressing a gene comprising the sequence as presented in SEQ ID NO:1 with the chemical compound, under conditions permitting expression of the gene; and (b) determining whether the proliferation or differentiation of the cell is stimulated or inhibited in the presence of the compound, as compared to a control and this invention further provides a method for identifying a test chemical compound that modulates proliferation or differentiation of cells which comprises the steps of (a) providing a mixture comprising polypeptide 76A11, (b) contacting said mixture with a test substance under conditions which normally lead to 76A11 activity, and (c) determining whether the activity of said polypeptide is affected by said compound.

### BACKGROUND OF THE INVENTION

1. Technical Field

The present invention relates to the identification of genes involved in proliferation and differentiation of embryonic stem cells. This application discloses a gene and a polypeptide, and negative regulators or modulators of the proliferation and differentiation pathways.

2. Background Art

Embryogenesis is the fundamental process of differentiation of all tissues from a fertilized egg. During this process, the cells of the developing embryo differentiate and raise their level of commitment, starting out as pluripotent cells, and ending up as fully differentiated mature cells. In mammals, the property of pluripotentiality is restricted to cells of the early embryo and to tissue-specific stem cells (for review, see Smith, A. 1998, Curr. Biol., 8:R802-R804).

Stem cells are characterized by two traits: 1) They have the capacity to produce daughter cells that are destined to differentiate and give rise to several different cell types and, therefore, they are pluripotent, and 2) they are self-renewing, i.e., they have the capacity to produce daughter cells that maintain the characteristics of the mother stem cell. This means that the stem cell population can be maintained or expanded indefinitely.

Over the past several years, the importance of stem cells for therapy in injury and disease states has grown. For example, these cells can be used to compensate for loss or death of cells with cellular dysfunction. The ability to stimulate proliferation and differentiation of stem cells in vivo is crucial for their use in medical and/or therapeutic procedures. Another application is isolation or generation of stem cells in vitro, and using them for transplantation, such as is done in bone marrow transplantation. In this case, the expansion of the stem cell fraction in the whole bone marrow, and induction of proliferation and differentiation after transplantation, can contribute to successful recovery.

Embryonic Stem Cells

A major step in realizing the above therapeutic goals was the establishment of mammalian embryonic stem cell (ES cell) lines that can be grown in culture. These cells are derived from the totipotent cells of the inner cell mass (ICM) in the early mammalian embryo and are capable of unlimited, undifferentiated proliferation in vitro. The first lines (developed in the early 1980's) were originally from mouse blastocysts (Andrews P W et al., 1984, Lab. Invest. 50:147-162; Evans, M J and Kaufman M., 1981, Nature 292:154-156). During the last decade, establishment of lines from other mammals, including primates, has led the way to the generation of human ES cells (for review, see Prelle K et al., 1999, Cells Tissues Organs 165(3-4):220-236).

ES cells are true pluripotent cells as they can differentiate into all cell types. The multipotency of embryonic stem cells is evident from the following:

1. When the undifferentiated cells are injected into the cavity of blastocysts and the injected blastocysts are implanted into pseudopregnant mice, chimeric mice develop (for reviews, see Bradley, A, 1987, in Robertson E J (ed.) Teratoma and embryonic stem cells: A practical approach. Oxford IRL Press:71-112; Capecchi, M R , 1989, Trends Genet. 5:70-76; Rossant, J and Joyner, A L , 1989, Trends Genet. 5:277-283). The injected ES cells contribute to all cell types, including the germ layer;

2. When ES cells are injected into syngeneic mice, teratocarcinoma tumors develop (Wobus, A M et al., 1984, Exp.Cell Res., 152:212-219). These tumors comprise cells of all three embryonic germ layers; and

3. When ES cells are allowed to differentiate in vitro, they differentiate into different cell types.

The ability to obtain fully differentiated cells from undifferentiated ES cells suggests that, in vitro, the cells progress through commitment steps that result at each stage in morphological and molecular modifications. Since ES cells differentiate into all cell types, we now have the tools to define commitment steps, and the means to isolate somatic stem cells.

Spontaneous Differentiation of ES Cells

The undifferentiated state of ES cells can be maintained by growing them attached to the plate, on feeder cells (embryonic mouse fibroblasts called STO cells) or on gelatin-coated plates. An important supplement of the growth medium is Leukemia Inhibiting Factor (LIF) that prevents their differentiation. When ES cells are grown without LIF and allowed to aggregate, they form small spheroid balls of cells called embryoid bodies (EBs). Soon after induction of differentiation they are called simple EBs (SEBs) and have an outer layer of large visceral and parietal endoderm cells. As they continue to grow, the SEBs develop into cystic EBs (CEBs) with fluid-filled cavities and an inner layer of ectoderm-like cells. CEBs include cells from the three germ layers—endoderm, mesoderm, and ectoderm—and can survive in culture for 21 days.

The differentiation of ES cells to EBs involves morphological changes and gene expression alterations. The mRNA from genes that are expressed in the ICM can be found in undifferentiated ES cells, while transcripts from genes that are markers for specific cell types are found in the developing EB. Genes like RFX-1, and proto-onocogenes like c-myc, are highly expressed in undifferentiated ES cells, and their expression level is repressed after differentiation to EBs. Another interesting group is that of genes that begin to be expressed after differentiation of ES cells (for review, see Dushnik-Levinson, M and Benvenisty, N, 1995, Biol. Neonate, 67:77-83). Because differentiation of ES cells reflects early embryonic development, tissue-specific genes begin to be expressed. For example, the globin genes represent the complete differentiation of the hematopoietic system. Morphological changes and the formation of blood islands accompany this process. Moreover, the order of initiation of expression of the globin genes during differentiation of ES cells mimics their expression during normal embryonic development.

For the reasons discussed above, in vitro differentiation of ES cells is used extensively as a model for early mammalian embryogenesis, gene function and development, as at this stage the embryo is very small and buried deep in the uterine wall (for review, see Wobus, A M and Boheler, K R (eds), 1999 Cells Tissues Organs, 165:129-130).

Non-spontaneous Differentiation of ES Cells

One of the interesting characteristics of ES cells is that specific factors can cause them to divert from their regular mode of differentiation and force them to preferentially differentiate into one particular lineage (for review, see Dushnik-Levinson, M and Benvenisty, N, 1995, Biol. Neonate, 67:77-83). For example, when ES cells are exposed to retinoic acid (RA) during the first days of differentiation, a shift occurs in favor of the neuronal lineage (Bain, G et al., 1996, Biochem. Biophys. Res. Commun. 223(3):691-694). Thus, different hormones and cytokines can be tested, and their function as factors that direct differentiation, can be revealed. Moreover, investigating the molecular changes that follow the addition of the factor can lead to the identification of pathways and of key genes in the differentiation process.

The lack of suitable donor organs and tissues has led researchers to generate cell type-specific somatic cells from ES cells by means of non-spontaneous differentiation, as a source for transplantation. For example, ES cell-derived glia precursors have been shown to migrate and differentiate normally after transplantation into the mouse brain (Brustle, O et al., 1999, 285:754-756; for review, see Boheler, K R and Fiszman, M Y, 1999, Cells Tissues Organs, 165:237-245). The availability of human ES cells and the possibility of generating autologous ES cells by nuclear transfer provide exciting perspectives for the treatment of human disease (for review, see Gearhart, J, 1998, Science, 282:1061-1062; Solter, D and Gearhart, J, 1999, Science, 283:1468-1470).

In summary, it is generally assumed that proliferation and differentiation of pluripotent embryonic stem cells require expression of genes that may encode growth factors, differentiation factors and/or signals, most of which are yet to be discovered. It was the aim of the inventors to identify genes encoding the proteins that direct these processes. An understanding of the molecular mechanisms that govern stem cell fate is of fundamental significance in cellular and developmental biology, and the capabilities arising from such knowledge have major biomedical applications.

### SUMMARY OF THE INVENTION

This application discloses a human gene and corresponding polypeptide of novel function. The gene is termed the 76A11 gene and the corresponding polypeptide is termed the 76A11 polypeptide. The application discloses proliferation and differentiation functions for this gene and polypeptide. The application further discloses use of the polypeptide in the expansion of stem cells and use of antagonists of the polypeptide in the treatment of patients suffering from cancer.

### DESCRIPTION OF THE DRAWINGS

Other advantages of the present invention can be readily appreciated, as the same becomes better understood by reference to the following detailed description when considered in connection with the accompanying drawings wherein:

FIG. 1 is a Northern blot analysis of poly A⁺ RNA from undifferentiated and differentiated ES cells, and STO cells. Poly A⁺ RNA samples (3 μg) from STO cells (1); pool of 3, 4, 7 and 9 days differentiated ES cells (2); and undifferentiated ES cells (3) were separated on formaldehyde gels, transferred to nylon membrane, and hybridized with a probe corresponding to the sequence of the mouse gene, MUS_{—}76A11.

FIG. 2 presents the sub-cellular localization of MUS_{—}76A11 using immunofluoresence of HeLa cells transfected with MUS_{—}76A11-Flag.

### DETAILED DESCRIPTION OF THE INVENTION

Generally, the present invention provides methods and factors for proliferation and differentiation of embryonic stem cells.

An embodiment of the present invention provides for regulator molecules that affect the level of activity of the peptide or polypeptide provided for by the present invention. A preferred embodiment of the present invention provides for antagonists (inhibitors) and methods for identifying such antagonists, wherein such antagonists reduce or prevent the function of the peptide or polypeptide provided for by the invention. In a more preferred embodiment of the present invention there are provided antagonists that are small molecules, preferably small organic molecules, and antibodies, including single chain antibodies, and antisense oligonucleotides, antisense DNA or RNA molecules, proteins, polypeptides and peptides, including peptido-mimetics and dominant negatives, and expression vectors. Small molecules are referred to in the instant invention as chemical compounds that generally have a molecular weight of less than 2000 daltons, more preferably less than 1000 daltons.

The present invention provides for an isolated polynucleotide having a nucleotide sequence as presented in SEQ ID NO:1, or having homology thereto, or a fragment thereof, preferably comprising at least 30 nucleotides, most preferably at least 40 or more nucleotides, or a polynucleotide having a sequence complementary to SEQ ID NO:1.

An embodiment of the present invention provides for an isolated polynucleotide, wherein the polynucleotide comprises the amino acid-encoding portion of the sequence, as shown in SEQ ID NO:1, wherein the amino acid-encoding sequence begins with the start codon ATG and ends with the stop codon TAG.

An embodiment of the present invention encompasses an isolated polypeptide having an amino acid sequence encoded by a polynucleotide having a nucleotide sequence as set forth in SEQ ID NO:1.

Alternatively, the present invention provides for a polypeptide, wherein the polypeptide has an amino acid sequence as shown in SEQ ID NO:2. This is the “76A11 polypeptide”, and this term comprises fragments and homologs of the polypeptide which have any one of the biological functions of the naturally occurring 76A11 polypeptide. The 76A11 polypeptide may or may not contain the initiator

methionine depicted in SEQ ID NO:2. The “76A11 polypeptide” term also includes those polypeptides having additional amino acid residues, up to 50 amino acid residues, more preferably up to 25 amino acid residues, most preferably up to 10 amino acid residues that do not alter the function of the original molecule attached to the polypeptide, preferably at the N′-terminus or C′-terminus. Preferably, the additional amino acid residues represent a Flag-Tag (Li, F et al., 2001, J. Biol. Chem., 276(50):47542-47549.

The biological functions of the naturally occurring 76A11 polypeptide are proliferation of embryonic stem cells, differentiation of embryonic stem cells and the ability of the polypeptide to promote expansion of stem cells, either embryonic or adult.

In one embodiment of the invention the polynucleotide, having SEQ ID NO:1, or a homolog or a complementary polynucleotide or a fragment thereof, is delivered via a gene expression vehicle for use in gene therapy. In a preferred embodiment, the gene therapy is ex vivo gene therapy or in vivo gene therapy. In a more preferred embodiment of the invention the gene expression vehicle is selected from a group consisting of a DNA viral vector, a recombinant viral vector and a retroviral vector. A yet more preferred embodiment of the present invention provides for a composition comprising said expression vehicle. An even more preferred embodiment of the invention provides said composition, wherein said composition comprises a pharmaceutically acceptable carrier.

The present invention provides for use of said polynucleotide as a marker for identifying cancerous cells.

A preferred embodiment of the present invention provides for a method of regulating a gene with a polynucleotide having a coding sequence as presented in SEQ ID NO:1, or of regulating a gene product encoded by a nucleotide sequence as shown in SEQ ID NO:1, wherein the method comprises applying a small molecule to a cell ex vivo or in vivo.

The present invention provides for therapeutic use of the 76A11 polypeptide, wherein said therapeutic use is directed towards expansion of adult somatic stem cells in vivo or ex vivo.

A further embodiment of the invention is directed towards expansion of adult somatic stem cells, wherein the stem cells are selected from a group consisting of, but limited by, hematopoietic, neuronal, liver, pancreatic beta-, muscle, mesenchymal, skin stem cells and osteoprogenitor cells.

Patent application numbers WO 01/53312, CN 1269412 and EP 624646 all discuss the polynucleotide and polypeptide of the instant invention. None of these publications provides any evidence that inhibition of the gene product can be useful in the treatment of a subject with cancer, nor that the gene product can be useful in stem cell expansion.

Yet another embodiment of the present invention provides for an antibody directed to an epitope presented by the 76A11 polypeptide.

An antibody, for the purpose of the present invention, is defined as monoclonal, polyclonal or recombinant, or a fragment thereof, such fragment selected from a list that includes, but is not limited to, single-chain Fv (scFv), Fab, F(ab′)₂, and Fv. In one embodiment of the present invention, a fragment of an antibody is any part of the original antibody molecule that retains the specific recognition activity of the full-sized molecule. Antibodies are discussed in more detail below.

A preferred embodiment of the invention provides for said antibody, wherein the antibody is conjugated to a detectable moiety selected from the group consisting of fluorescent, metallic, enzymatic and radioactive markers.

A more preferred embodiment of the invention provides for said antibody, wherein the marker is selected from the group consisting of biotin, gold, ferritin, alkaline phosphatase, β-galactosidase, peroxidase, urease, fluorescein, rhodamine, tritium, ¹⁴C and iodinated compounds.

A molecule that comprises the antigen-binding portion of an antibody specific for a polypeptide, variant or fragment of the invention is also contemplated.

An embodiment of the present invention provides for use of said antibody for detection of cancerous cells or of undifferentiated ESC. A more preferred embodiment of the invention provides for a composition comprising said antibody. A yet more preferred embodiment of the invention provides for a composition comprising an antibody and a pharmaceutically acceptable carrier.

An embodiment of the present invention provides for said polynucleotide to be used as a probe for identifying undifferentiated embryonic stem cells.

A further embodiment of the present invention provides for said polynucleotide to be used as a probe for identifying undifferentiated adult stem cells.

An embodiment of the present invention provides for a differentiation factor or a proliferation factor encoded by the DNA sequence as presented in SEQ ID NO:1.

A preferred embodiment of the present invention provides for use of said differentiation factor for determination of the differentiation stage of selected cells.

A more preferred embodiment of the invention provides for use of said proliferation factor for the identification of proliferating cells from selected tissues.

The present invention provides for a method of preventing, treating or controlling disease characterized by aberrant proliferation or aberrant differentiation, comprising administering the 76A11 polypeptide or an antagonist thereof.

A preferred embodiment of the invention provides for a method for preventing, treating or controlling cancer, comprising administering an antagonist of the 76A11 polypeptide.

The present invention further provides for a method for advancing research in or for furthering studies of aberrant differentiation or aberrant proliferation of cells in the body of a mammal, comprising use of said polynucleotide or said polypeptide or antagonists thereof.

The present invention provides for a method of treating a cancer in a subject which comprises administering to the subject a therapeutically effective amount of an antagonist of a gene with a polynucleotide sequence as presented in SEQ ID NO:1 or of a gene product of said polynucleotide. In a preferred embodiment of the invention, the antagonist is an antibody.

In a more preferred embodiment of the invention, the antagonist is administered in conjunction with a chemotherapeutic agent. Examples of such chemotherapeutic agents include etoposide, 5-FU, cis-platin, doxorubicin, a Vinca alkaloid, taxol, cyclophosphamide, ifosphamide, chlorambucil, busulfan, mechlorethamine, mitomycin, dacarbazine, carboplatin, thiotepa, daunorubicin, idarubicin, mitoxantlirone, bleomycin, esperamicin A₁, dactinomycin, plicamycin, carmustine, lomustine, tauromustine, streptozocin, melphalan, dactinomycin, and procarbazine.

By “in conjunction with” is meant that the two agents, chemotherapeutic agent and 76A11 antagonist, are administered to the patient simultaneously, in the same drug composition or in separate drug compositions mixed together. Alternatively, the two agents are administered separately, in either temporal order.

The present invention provides for a method for identifying a chemical compound that modulates proliferation or differentiation of cells, which comprises:

(a) contacting cells expressing a gene of the sequence as presented in SEQ ID NO:1 with the chemical compound, under conditions permitting expression of the gene, and

(b) determining whether the proliferation or differentiation of the cell is stimulated or inhibited in the presence of the compound, as compared to a control.

Yet a further embodiment of the invention provides for a method of preparing a pharmaceutical composition which comprises determining whether a chemical compound stimulates or inhibits the herein-disclosed proliferation or differentiation gene by:

(i) using said screening method, and

(ii) admixing said compound with a pharmaceutically acceptable carrier.

The present invention further provides for a method of screening a plurality of chemical compounds not known to modulate proliferation or differentiation, in order to identify a compound that modulates proliferation or differentiation, which comprises:

(a) contacting cells expressing a gene comprising a sequence as presented in SEQ ID NO:1 with the plurality of chemical compounds not known to stimulate or inhibit proliferation or differentiation, under conditions permitting expression of the gene;

(b) determining whether the proliferation or differentiation of the cell is stimulated or inhibited in the presence of the compounds, as compared to a control; and, if so

(c) separately determining whether the stimulation or inhibition of proliferation or differentiation is increased by each compound included in the plurality of compounds, so as to thereby identify the compound which stimulates or inhibits the proliferation or differentiation.

A further embodiment of the invention provides for said method of screening a plurality of chemical compounds, wherein said cell in said contacting step has been transfected by the gene being expressed.

In a more preferred embodiment of the invention, said chemical compound is prepared as a pharmaceutical composition. In yet a more preferred embodiment of the invention, the pharmaceutical composition is administered in conjunction with a chemotherapeutic agent. Examples of such chemotherapeutic agents include etoposide, 5-FU, cis-platin, doxorubicin, a Vinca alkaloid, taxol, cyclophosphamide, ifosphamide, chlorambucil, busulfan, mechlorethamine, mitomycin, dacarbazine, carboplatin, thiotepa, daunorubicin, idarubicin, mitoxanthrone, bleomycin, esperamicin A₁, dactinomycin, plicamycin, carmustine, lomustine, tauromustine, streptozocin, melphalan, dactinomycin, and procarbazine.

An embodiment of the invention provides for a method for identifying a test chemical compound that modulates proliferation or differentiation of cells that comprises the steps of:

a. measuring the activity of the human 76A11 polypeptide, or a fragment thereof having proliferation or differentiation activity,

b. providing a mixture comprising polypeptide 76A11,

c. contacting said mixture with a test substance under conditions that normally lead to 76A11 activity, and

d. determining whether the activity of said polypeptide or fragment is affected by said compound.

A further embodiment of the invention provides for the chemical compound to be prepared as part of a pharmaceutical composition. Yet a further embodiment of the invention provides for said pharmaceutical composition to be administered in conjunction with a chemotherapeutic agent. Examples of such chemotherapeutic agents include etoposide, 5-FU, cis-platin, doxorubicin, a Vinca alkaloid, taxol, cyclophosphamide, ifosphamide, chlorambucil, busulfan, mechlorethamine, mitomycin, dacarbazine, carboplatin, thiotepa, daunorubicin, idarubicin, mitoxanthrone, bleomycin, esperamicin A₁, dactinomycin, plicamycin, carmustine, lomustine, tauromustine, streptozocin, melphalan, dactinomycin, and procarbazine.

Additional embodiments of the present invention include dominant negative peptides that compete with the biological activity of the 76A11 polypeptide. Further embodiments of the invention include, but are not limited to, isolated antisense oligonucleotides.

More in particular, the invention further comprehends isolated and/or purified polynucleotides and isolated and/or purified polypeptides having at least about 70%, preferably at least about 75%, more preferably at least about 80%, even more preferably at least about 90%, most preferably at least about 95% homology, or even about 98%, 99% or 100% homology to the polynucleotides and polypeptides disclosed herein. The invention also comprehends that these homologous polynucleotides and polypeptides can be used in the same fashion as the herein or aforementioned polynucleotides and polypeptides.

The present invention is additionally directed to pharmaceutical compositions that include the nucleic acids, proteins or polypeptides in accordance with the present invention, along with pharmaceutically acceptable carriers or excipients.

In addition, the present invention is directed to knockout or transgenic non-human animals, in which a gene identified by the present invention has been knocked out or introduced. Transgenic non-human animals can be utilized in assays designed to screen antagonist molecules of the polynucleotide and/or polypeptide identified in the present invention.

Methods

General methods in molecular biology: Standard molecular biology techniques known in the art and not specifically described were generally followed, as in Sambrook et al., Molecular Cloning: A Laboratory Manual, Cold Spring Harbor Laboratory Press, New York (1989), in Ausubel et al., Current Protocols in Molecular Biology, John Wiley and Sons, Baltimore, Md. (1989), in Perbal, A Practical Guide to Molecular Cloning, John Wiley & Sons, New York (1988), in Watson et al., Recombinant DNA, Scientific American Books, New York, in Birren et al. (eds) Genome Analysis: A Laboratory Manual Series, Vols. 1-4 Cold Spring Harbor Laboratory Press, New York (1998) and methodology as set forth in U.S. Pat. Nos. 4,666,828; 4,683,202; 4,801,531; 5,192,659 and 5,272,057 and incorporated herein by reference. Polymerase chain reaction (PCR) was carried out generally as in PCR Protocols: A Guide to Methods and Applications, Academic Press, San Diego, Calif. (1990). In-situ (In-cell) PCR in combination with flow cytometry can be used for detection of cells containing specific DNA and mRNA sequences (Testoni et al., 1996, Blood 87:3822)

For Gene Therapy

Gene therapy as used herein refers to the transfer of genetic material (e.g., DNA or RNA) of interest into a host to treat or prevent a genetic or acquired disease or condition phenotype. The genetic material of interest encodes a product (e.g., a protein, polypeptide, peptide, functional RNA, antisense) the production in vivo of which is desired. For example, the genetic material of interest can encode a hormone, receptor, enzyme, polypeptide or peptide of therapeutic value. Alternatively, the genetic material of interest encodes a suicide gene. For a review see, in general, the text: Gene Therapy, Advances in Pharmacology 40, Academic Press, 1997.

Two basic approaches to gene therapy have evolved: (1) ex vivo and (2) in vivo gene therapy. In ex vivo gene therapy, cells are removed from a patient and, while being cultured, are treated in vitro. Generally, a functional replacement gene is introduced into the cell via an appropriate gene delivery vehicle/method (transfection, transduction, homologous recombination, etc.) and an expression system as needed, and then the modified cells are expanded in culture and returned to the host/patient. These genetically re-implanted cells have been shown to express the transfected genetic material in situ.

In in vivo gene therapy, target cells are not removed from the subject; rather, the genetic material to be transferred is introduced into the cells of the recipient organism in situ, e.g., within the recipient. In an alternative embodiment, if the host gene is defective, the gene is repaired in situ (Culver, February 1998, (Abstract) Antisense DNA & RNA based therapeutics, Coronado, Calif.). These genetically altered cells have been shown to express the transfected genetic material in situ.

Vectors can be introduced into cells or tissues by any one of a variety of known methods in the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995), Vectors: A Survey of Molecular Cloning Vectors and Their Uses, Butterworths, Boston Mass. (1988), and Gilboa et al. (1986) BioTechniques 4(6):504-512, and include, for example, stable or transient transfection, lipofection, electroporation and infection with recombinant viral vectors. In addition, see U.S. Pat. Nos. 5,464,764 and 5,487,992 for positive-negative selection methods.

The expression product generated by vectors or recombinants can also be isolated and/or purified from infected or transfected cells; for instance, to prepare compositions for administration to patients. However, in certain instances, it may be advantageous to not isolate and/or purify an expression product from a cell, for instance, when the cell or portions thereof enhance the effect of the polypeptide.

As used herein, “polypeptide” refers to a stretch of amino acids joined covalently by peptide bonds. What are commonly referred to as peptides, polypeptides and proteins, dependent on the length of the amino acid chain, are all included in the definition of polypeptide for the purposes of the present invention.

As used herein, “treatment” refers to clinical intervention in an attempt to alter the natural course of the individual or cell being treated, and may be performed either for prophylaxis or during the course of clinical pathology. Desirable effects of the treatment include preventing occurrence or recurrence of disease, alleviation of symptoms, diminishment of any direct or indirect pathological consequences of the disease, preventing metastases, decreasing the rate of disease progression, amelioration or palliation of the disease state, and remission or improved prognosis.

An inventive vector or recombinant nucleotide expressing a gene or a portion thereof identified herein or from a method herein can be administered in any suitable amount to achieve expression at a suitable dosage level, e.g., a dosage level analogous to the herein mentioned dosage levels (wherein the gene product is directly present). The inventive vector or recombinant nucleotide can be administered to a patient or infected or transfected into cells in an amount of about at least 10³ pfu; more preferably about 10⁴ pfu to about 10¹⁰ pfu, e.g., about 10⁵ pfu to about 10⁹ pfu, for instance about 10⁶ pfu to about 10⁸ pfu. In plasmid compositions, the dosage should be a sufficient amount of plasmid to elicit a response analogous to compositions wherein gene product or portion thereof is directly present; or to have expression analogous to dosages in such compositions; or to have expression analogous to expression obtained in vivo by recombinant compositions. For instance, suitable quantities of plasmid DNA in plasmid compositions can be 1 g to 100 mg, preferably 0.1 to 10 mg, e.g., 500 g, but lower levels such as 0.1 to 0.2 mg or preferably even lower levels such as 1-10 g may be employed. Documents cited herein regarding DNA plasmid vectors can be consulted for the skilled artisan to ascertain other suitable dosages for DNA plasmid vector compositions of the invention, without undue experimentation.

Delivery of gene products (products from herein defined gene: gene identified herein or by inventive methods or portions thereof) and/or antibodies or portions thereof and/or agonists or antagonists thereof (collectively or individually “therapeutics”), and compositions comprising the same, as well as of compositions comprising a vector expressing gene products, can be done without undue experimentation from this disclosure and the knowledge in the art.

Antibody Production

Antibodies may be used in various aspects of the invention, e.g., in detection or treatment or prevention methods. Antibodies can be monoclonal, polyclonal or recombinant for use in the immunoassays or other methods of analysis necessary for the practice of the invention. Conveniently, the antibodies may be prepared against the immunogen or antigenic portion thereof, for example, a synthetic peptide based on the sequence, or prepared recombinantly by cloning techniques or the natural gene product and/or portions thereof may be isolated and used as the immunogen. The genes are identified as set forth in the present invention and the gene product identified. Immunogens can be used to produce antibodies by standard antibody production technology well known to those skilled in the art, as described generally in Harlow and Lane (1988) Antibodies: A Laboratory Manual, Cold Spring Harbor Laboratory, Cold Spring Harbor, N.Y.; and Borrebaeck (1992) Antibody Engineering—A Practical Guide, W. H. Freeman and Co. Antibody fragments can also be prepared from the antibodies and include Fab, F(ab')₂, Fv and scFv prepared by methods known to those skilled in the art (Bird et al., 1988, Science 242:423-426). Any peptide having sufficient flexibility and length can be used as an scFv linker. Usually the linker is selected to have little or no immunogenicity. An example of a linking peptide is (GGGGS)₃, which bridges approximately 3.5 nm between the C-terminus of one V region and the N-terminus of another V region. Other linker sequences can be used, and can provide additional functions, such as a means for attaching a drug or a solid support.

For producing polyclonal antibodies a host, such as rabbit or goat, is immunized with the immunogen or immunogen fragment, generally with an adjuvant and, if necessary, coupled to a carrier; and antibodies to the immunogen are then collected from the sera. Further, the polyclonal antibody can be absorbed such that it is monospecific, i.e., the sera can be absorbed against related immunogens so that no cross-reactive antibodies remain in the sera, thus rendering it monospecific.

For producing monoclonal antibodies the technique involves hyperimmunization of an appropriate donor with the immunogen, generally a mouse, and isolation of splenic antibody-producing cells. These cells are fused to a cell displaying immortality, such as a myeloma cell, to provide a fused cell hybrid which is immortal and which secretes the required antibody. The cells are then cultured in bulk, and the monoclonal antibodies are harvested from the culture media for use.

For producing recombinant antibody (see generally Huston, J S et al., 1991, Methods Enzymol, 203:46-88; Johnson and Bird, 1991, Methods Enzymol, 203:88-99; Mernaugh and Mernaugh, 1995, in Molecular Methods In Plant Pathology, R P Singh and U S Singh (eds.); CRC Press Inc., Boca Raton, Fla.:359-365), messenger RNAs from antibody producing B-lymphocytes of animals or from hybridoma are reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody or antibody fragment is expressed using a suitable expression system to obtain recombinant antibody. Antibody cDNA can also be obtained by screening pertinent expression libraries.

The antibody can be bound to a solid support substrate or conjugated with a detectable moiety or be both bound and conjugated, as is well known in the art. (For a general discussion of conjugation of fluorescent or enzymatic moieties, see Johnstone & Thorpe, Immunochemistry in Practice, Blackwell Scientific Publications, Oxford, 1982.) The binding of antibodies to a solid support substrate is also well known in the art (for a general discussion, see Harlow & Lane, Antibodies: A Laboratory Manual, Cold Spring Harbor Laboratory Publications, New York, 1988 and Borrebaeck, Antibody Engineering—A Practical Guide, W. H. Freeman and Co., 1992). The detectable moieties contemplated with the present invention can include, but are not limited to, fluorescent, metallic, enzymatic and radioactive markers such as biotin, gold, ferritin, alkaline phosphatase, β-galactosidase, peroxidase, urease, fluorescein, rhodamine, tritium, ¹³C and iodination.

An antibody can also be used as an active agent in a therapeutic composition and such antibodies can be humanized, for instance, to enhance their effect (Huls et al., 1999, Nature Biotech. 17). “Humanized” antibodies are antibodies in which at least part of the sequence has been altered from its initial form to render it more like human immunoglobulin. In one version, the H chain and L chain C regions are replaced with human sequence. In another version, the CDR regions comprise amino acid sequences from the antibody of interest, while the V framework regions have been converted to human sequences. See, for example, EP 0 329 400. In a third version, V regions are humanized by designing consensus sequences of human and mouse V regions, and converting residues outside the CDRs that are different from the consensus sequences. The invention encompasses all the above-described antibodies, including humanized mAbs and also human mAbs.

Completely human antibodies are particularly desirable for therapeutic treatment of human patients. Human antibodies can be made by a variety of methods known in the art, including phage display methods using antibody libraries derived from human immunoglobulin sequences (see also, U.S. Pat. Nos. 4,444,887 and 4,716,111; and PCT publications WO 98/46645, 98/50433, WO 98/24893, WO 98/16654, WO 96/34096, WO 96/33735, and WO 91/10741, each of which is incorporated herein by reference in its entirety).

Additional information regarding all types of antibodies, including humanized antibodies, human antibodies and antibody fragments can be found in WO 01/05998, which is incorporated herein by reference in its entirety.

The present invention also provides a composition of the isolated nucleic acid molecule, a vector comprising the isolated nucleic acid molecule, a composition containing said vector and a method for preventing, treating or controlling cancer, comprising administering the inventive composition, or the inventive vector, and a method for preparing a polypeptide comprising expressing the isolated nucleic acid molecule or comprising expressing the polypeptide from the vector.

In a preferred embodiment of the invention cancer includes, but is not limited to, leukemia, melanoma, sarcoma, and myeloma.

As used herein, the term “subject,” “patient,” and/or “host” include, but are not limited to human, bovine, pig, mouse, rat, goat, sheep, horse and other mammals.

Those skilled in the art will recognize that the components of the composition should be selected to be chemically inert with respect to the gene product and optional adjuvant or additive. This will present no problem to those skilled in chemical and pharmaceutical principles, or problems can be readily avoided by reference to standard texts or by simple experiments (not involving undue experimentation), from this disclosure and the documents cited herein.

Recombinant Protein Purification

Protein purification is practiced as is known in the art as described in, for example, Marshak et al., “Strategies for Protein Purification and Characterization. A laboratory course manual.” CSHL Press (1996).

Small Molecules

In an embodiment of the present invention, a small molecule is administered to a target cell, tissue, or organism, such that the small molecule permeates the cell membrane of said target cell, or of the cell in the target tissue or organism and effects an activation or inactivation of a specified polypeptide therein.

In a preferred embodiment of the present invention, the small molecule is an antagonist of the target polypeptide. The compounds to be administered comprise inter alia small chemical molecules, as well as antibodies or fragments thereof, including single chain antibodies, antisense oligonucleotides, antisense DNA or RNA molecules, polypeptides, including peptido-mimetics and dominant negatives, and expression vectors.

Dominant negative (or negative dominant) peptide or polypeptide refers to a peptide or polypeptide encoded by a partial cDNA sequence that encodes for a part of a protein (see Herskowitz, 1987, Nature, 329:219-222). This peptide can have a different function from the protein from which it was derived. It can interact with the full protein and inhibit its activity or it can interact with other proteins and inhibit their activity in response to the full protein. Dominant negative means that the peptide is able to overcome the natural proteins and fully inhibit their activity to give the cell a different characteristic, like resistance or sensitization to killing. For therapeutic intervention either the peptide itself is delivered as the active ingredient of a pharmaceutical composition or the cDNA can be delivered to the cell utilizing the same methods used for antisense delivery. An antisense therapeutic construct can be delivered to the cells and can be rendered nuclease resistant as is known in the art (Agrawal, 1996, TIBTECH, 14:376; Calabretta et al., 1996, Semin. Oncol. 23:78; Crooke, 1995, Hematol. Pathol. 2:59; Felgner, 1997, Scientific American. June, 1997:102-106; Gewirtz, 1993, Stem Cells Dayt. 11:96; Hanania et al., 1995, Am. J. Med. 99:537; Lefebvre-d'Hellencourt et al., 1995, Eur. Cytokine Netw. 6:7; Lev-Lehman et al., 1997, In Antisense Therapeutics, A. Cohen and S. Smicek (eds), Plenum Press, New York; Loke et al., 1989, PNAS (USA) 86:3474; Wagner et al., 1996, Nature Biotechnology 14:840-844; Wagner, 1994, Nature 372:333; Radhakrishnan et al., 1990, J. Org. Chem. 55:4693-4699).

Approaches have recently been developed that utilize small molecules that can bind directly to proteins and can be used to alter protein function (see review, B. R. Stockwell, 2000, Nature Reviews/Genetics, 1:116-125). Low molecular weight organic compounds can permeate the plasma membrane of target cells relatively easily and, therefore, methods have been developed for their synthesis. These syntheses, in turn, have yielded libraries that contain ligands for many proteins. Recent developments have brought a greatly increased variety of creatively selected, novel, small organic molecules that will function as powerful tools for perturbing biological systems. Such small molecules can be used to activate or inactivate specific members of a protein family. Small molecules generally have a molecular weight of less than 2000 Dalton, more preferably less than 1000 Dalton.

The compounds of the present invention (e.g. small molecules, polypeptides) are administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

A compound of the present invention can be administered in various ways. It should be noted that it can be administered as the compound or as a pharmaceutically acceptable salt and can be administered alone or as an active ingredient in combination with pharmaceutically acceptable carriers, diluents, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. The patient being treated is a warm-blooded animal and, in particular, a mammal including man. The pharmaceutically acceptable carriers, diluents, adjuvants and vehicles as well as implant carriers generally refer to inert, non-toxic solid or liquid fillers, diluents or encapsulating material not reacting with the active ingredients of the invention.

It is noted that humans are treated generally longer than the mice or other experimental animals exemplified herein. The doses can be single doses or multiple doses over a period of several days, but single doses are preferred. The treatment generally has a length proportional to the'length of the disease process and drug effectiveness and the patient species being treated. Thus, one can scale up from animal experiments, e.g., rats, mice, and the like, to humans, by teciniques from this disclosure and knowledge in the art, without undue experimentation.

When administering the compound of the present invention parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Non-aqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including anti-microbial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used has to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicles, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compounds in tablets, suspensions, solutions, emulsions, capsules, powders, syrups and the like are usable. Known techniques that deliver the formulation orally or intravenously and retain the biological activity are preferred. In one embodiment, the compound of the present invention can be administered initially by intravenous injection to bring blood levels to a suitable level. The patient's blood levels are then maintained by an oral dosage form, although other forms of administration, dependent upon the patient's condition and as indicated above, can be used. The quantity to be administered varies for the patient being treated and varies from about 100 ng/kg of body weight to 100 mg/kg of body weight per day, and preferably is from 10 μg/kg to 10 mg/kg per day.

Aims of the Study

Clinical Applications of the ESC Newly Identified Proliferation/Differentiation Factors

One of the main characteristics of malignant tumors is the high proliferation rate of the transformed cells. Genes that can be cell type-specific or non-specific govern the division time of the cell. Many oncogenies (which are involved in malignant transformation) are expressed in the undifferentiated state of ESC and are down-regulated during the differentiation pathways. This finding is consistent with the high proliferation rate of undifferentiated ESC. Taken together, the gene (polynucleotide) isolated from the work described herein may take part in the development of cancer. Furthermore, because ESC are in the undifferentiated state, the gene is more likely to be related to cell type non-specific transformation. Therefore, the genes (polynucleotides) disclosed herein may be used as markers for cancerous cells, and these genes (or a portion of them comprising at least 30 nucleotides, preferably at least 40 or more nucleotides) may be used as probes to identify cancerous cells. The genes disclosed herein might also be used as markers for undifferentiated ESC, and these genes (or a portion of them comprising at least 30 nucleotides, preferably at least 40 or more nucleotides) may be used as probes to identify undifferentiated ESC.

Furthermore, the genes disclosed herein may be inserted into an expression vector by methods known in the art, as described above (e.g., Sambrook et al., Molecular Cloning: A Laboratory Manual, Cold Spring Harbor Laboratory Press, New York (1989); Ausubel et al., Current Protocols in Molecular Biology, John Wiley and Sons, Baltimore, Md. (1989); Perbal, A Practical Guide to Molecular Cloning, John Wiley & Sons, New York (1988)) to produce the corresponding polypeptide. These polypeptides can be used therapeutically for expansion of adult somatic stem cells ex vivo or in vivo. Expansion of stem cells can be performed on any stem cells obtainable from the mammalian body. Stem cells can, for example, be selected from a group consisting of hematopoietic, neuronal, liver, pancreatic beta-, muscle, mesenchymal, skin stem cells and osteoprogenitor cells.

Additionally, antibodies can be produced to these polypeptides by methods known in the art (as described above), and these antibodies can be used for detection of cancerous cells and undifferentiated ESC.

Tissue engineering is an emerging field, still in its infancy. In this process, cells are grown in vitro on a scaffold support and then transplanted to the patient's body. The growth medium must include growth factors that direct differentiation and maintenance of the cells. In some cases, the cells are grown on a feeder cell layer that provides the suitable growth factors. The success of this new medical approach largely depends upon the ability to understand the complex cellular interactions, intervening with the right scaffold material and the exact growth factors. Large varieties of genes that are related to differentiation are expressed during ESC differentiation. Those genes represent differentiation to all the cell types of the body. Isolation of those genes contributes to the long procedure of making tissue engineering accessible for many cell types.

In summary, the isolated genes (polynucleotides) are connected to the development and differentiation of tissues, or to general cell proliferation. The identification of key genes that are involved in these crucial processes can be beneficial for new drug development.

More in particular, with respect to polynucleotides and polypeptides expressed from them, the invention further comprehends isolated and/or purified polynucleotides and isolated and/or purified polypeptides having homology to the purified polynucleotide or the polypeptide of the invention. The invention also comprehends that these polynucleotides and polypeptides can be used in the same fashion as the herein or aforementioned polynucleotides and polypeptides.

Nucleotide sequence homology can be determined using the “Align” program of Myers and Miller, ((1988) CABIOS 4:11-17) and available at NCBI. Alternatively or additionally, the term “homology”, for instance with respect to a nucleotide or amino acid sequence, can indicate a quantitative measure of homology between two sequences. The percent sequence homology can be calculated as (Nref−Ndif)*100/Nref, wherein Ndif is the total number of non-identical residues in the two sequences when aligned and wherein Nref is the number of residues in one of the sequences. Hence, the DNA sequence AGTCAGTC has a sequence similarity of 75% to AATCAATC (Nref=8; Ndif=2).

Alternatively or additionally “homology”, with respect to sequences, can refer to the number of positions with identical nucleotides or amino acid residues divided by the number of nucleotides or amino acid residues in the shorter of the two sequences, wherein alignment of the two sequences can be determined in accordance with the Wilbur and Lipman algorithm ((1983) Proc. Natl. Acad. Sci. USA 80:726), for instance, using a window size of 20 nucleotides, a word length of 4 nucleotides, and a gap penalty of 4, and computer-assisted analysis and interpretation of the sequence data including alignment, can be conveniently performed using commercially available programs (e.g., Intelligenetics™ Suite, Intelligenetics Inc., CA). When RNA sequences are said to be similar, or to have a degree of sequence identity or homology with DNA sequences, thymidine (T) in the DNA sequence is considered equal to uracil (U) in the RNA sequence. RNA sequences within the scope of the invention can be derived from DNA sequences or their complements, by substituting thymidine (T) in the DNA sequence with uracil (U).

Additionally or alternatively, amino acid sequence similarity or identity or homology can be determined, for instance, using the BlastP program (Altschul et al., Nucl. Acids Res. 25:3389-3402) and available at NCBI. The following references provide algorithms for comparing the relative identity or homology of amino acid residues of two proteins, and additionally, or alternatively, with respect to the foregoing, the teachings in these references can be used for determining percent homology or identity: Smith et al., 1981, Adv. Appl. Math. 2:482-489; Smith et al., 1983, Nucl. Acids Res. 11:2205-2220; Devereux et al., 1984, Nucl. Acids Res. 12:387-395; Feng et al., 1987, J. Molec. Evol. 25:351-360; Higgins et al., 1989, CABIOS 5:151-153; and Thompson et al., 1994, Nucl. Acids Res. 22:4673-4680.

Polynucleotide sequences that are complementary to any of the sequences or fragments encompassed by the present invention discussed above are also considered to be part of the present invention. Whenever any of the sequences discussed above are produced in a cell, the complementary sequence is concomitantly produced and, thus, the complementary sequence can also be used as a probe for the same diagnostic purposes.

Initial isolation of the gene discussed herein was performed in a mouse system. Subsequent work allowed for the identification and isolation of the human nucleic acid sequence equivalent (homolog). Techniques for obtaining the human homolog of an isolated mouse gene are well known in the art and are discussed more fully in the Examples that follow, especially Example 2. For mouse and human genes to be considered homologs, they need to have some minimum of homology between them. It is worth noting that, regardless of whether or not one knows the actual sequence of the corresponding human gene, the mouse gene has utility as a probe for seeking and identifying the corresponding human gene which, when identified, will have its own utility.

Homology at the amino acid level between the human and mouse counterparts of the gene presented in the instant invention is approximately 80%. A database for mouse-human gene sequence comparisons has been compiled and is constantly being updated (Blake J A, Eppig J T, Richardson J E, Bult C J, Kadin J A, and the Mouse Genome Database Group. 2001. The Mouse Genome Database (MGD): Integration Nexus for the Laboratory Mouse. Nucleic Acids Res 29:91-94.). This emphasizes the volume of work that is being performed in the field and the applicability of studies of the mouse genome to the study of human genetic counterparts.

Similarly, the work described herein was performed with an in situ mouse model. Mouse models are becoming increasingly accepted as appropriate systems for studying specific cancers and potential methods of treatment thereof in humans (e.g., Hochman J, et al., 2001, Cancer Res, 61:5242-5247; Ludwig T, et al., 2001, Oncogene, 20:3937-3948; Shaker M R et al., 2000, Clin Exp Metastasis, 18:429-438; for a review, see Anisimov V N, 2001, Mech Ageing Dev, 122:1221-1255).

Thus, whereas the initial work was performed in mouse, there exists a substantial body of data supporting mouse-human genetic data comparisons, as well as murine models for studying human cancers.

Polypeptide Encoded by the Gene Described Above

Once the sequence of any full-length cDNA is obtained, the polypeptide encompassed thereby is readily determinable by analysis of the sequence to find the start and stop codons and by then decoding the amino acid sequence encoded by the cDNA. Thus, the present invention also encompasses any polypeptide encoded by a full-length cDNA encompassed by the present invention as discussed above. The amino acid sequence of such a polypeptide is presented in SEQ ID NO:2. Such polypeptide will be differentially expressed. This polypeptide can be used to raise antibodies that could be used in such a diagnostic assay for the presence of such a protein.

Analogs of a polypeptide encoded by the DNA sequence discovered in the assay described herein are also comprehended by the present invention. Preferably, the analog is a variant of the native sequence that has an amino acid sequence having at least 70% homology to the native amino acid sequence and retains the biological activity thereof. More preferably, such a sequence has at least 85% homology, at least 90% homology or, most preferably, at least 95%, 97%, 99% or even 100% homology to the native sequence.

In a preferred embodiment of the invention, a medicament or treatment can be devised based on the gene identified herein. Alternatively, or additionally, the medicament or treatment can be the particular polypeptide expressed by the gene detected in the inventive methods, or that which inhibits that polypeptide, e.g., binds to it. Similarly, additionally, or alternatively, the medicament or treatment can be a vector which expresses the polypeptide expressed by the gene detected in the inventive methods or that which inhibits expression of that gene; again, for instance, that which can bind to it and/or otherwise prevents its transcription or translation. The selection of administering a polypeptide or that which expresses it, or of administering that which inhibits the polypeptide or the gene expression, can be performed without undue experimentation, e.g., based on regulation of the various steps, beginning with gene expression and ending with polypeptide activity, as determined by inventive methods.

By “regulation” or “modulation” is meant increasing or decreasing the translation of a target nucleic acid into the respective polypeptide or increasing or decreasing the rate of expression of a target protein in a target cell or increasing or decreasing the activity of the protein in a target cell.

A positively identified polynucleotide sequence is an EST. The location of an EST in a full-length cDNA is determined by analyzing the EST for the presence of coding sequence(s). A conventional computer program is used to predict the extent and orientation of the coding region of a sequence (using all six reading frames). Based on this information, it is possible to infer the presence of start or stop codons within a sequence and to determine whether the sequence is completely coding or completely non-coding or a combination of the two. It should be noted that both coding and non-coding regions may provide ESTs equally useful in the described invention.

### EXAMPLE 1

### Isolation of Genes Involved in Proliferation and/or Differentiation

Methodology

A cDNA micro-array (MUS chip) was prepared according to the sequence-dependent gene identification (SDGI) method, using RNA extracted according to standard protocols. The SDGI method is described in co-assigned PCT application PCT/US01/09392, filed Mar. 23, 2001, which is incorporated herein by reference.

A selection of RNA samples used in preparation of the micro-array was obtained from ESC that were undifferentiated, spontaneously differentiated, or differentiated in culture. Samples were prepared over a differentiation course of ten days.

After identification and sequencing of up-regulated genes obtained using this system, the proprietary sequence analysis tools of the assignee were utilized for annotation and extension mining of sequence information. Analysis of the available biomedical literature relating to these statistically significant genes allowed for choice of appropriate candidates for further research.

Validation of Selected Candidate Gene

Initial verification of the expression results of the candidate gene was carried out by Northern blot hybridization, RT-PCR and in situ studies. Candidate genes for specific differentiation pathways are currently being examined in vitro for their ability to form distinct lineage after transfection to mouse ES cells. In addition, candidate genes involved in proliferation/cancer were also examined in vitro for their ability to increase proliferation rate of mouse ESC or other cell lines after transfection.

Conclusive in vivo validation of the candidate gene is performed by transplantation of the stable undifferentiated ES cells to mouse to test for teratoma formation and determination of teratoma composition (different lineages) and rate of tumor growth. In addition, direct injection of the “candidate drug”, i.e., polypeptide or portion thereof encoded by such gene, is performed in mice, testing for the desired therapeutic effect (using different disease models).

For “cancer-related” genes such as 76A11, transgenic mice are in preparation in order to evaluate spontaneous tumor formation.

Results

General Parameters for Selection of Pre-candidates

Pre-canididates were selected according to several parameters:

1. Length of sequence: greater than 150 bp

2. Known sequences or sequences with homology to ESTs that resemble known genes that have not previously been associated with the functions described herein.

3. Differential in the microsomal hybridization: above background value, in favor of secreted and membrane protein product

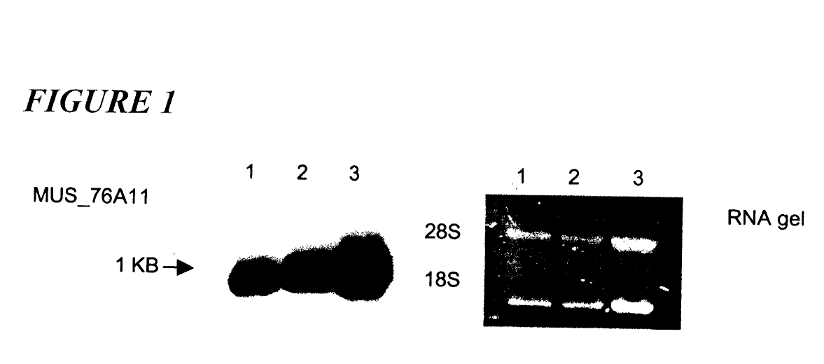

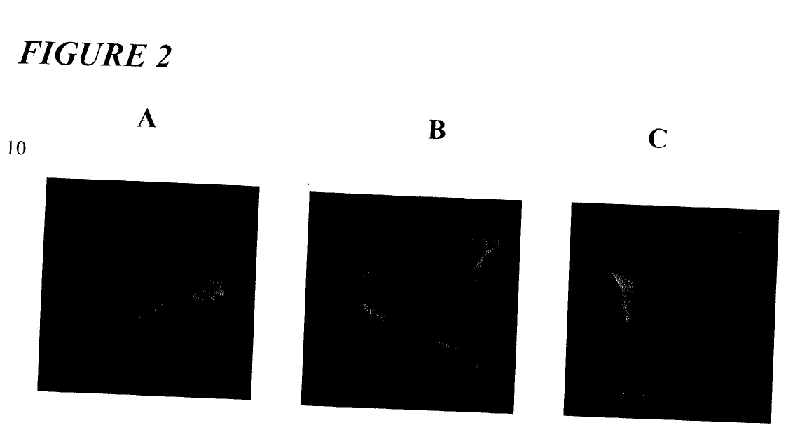

The murine pre-candidate homolog was isolated and subjected to Northern blot hybridization in order to verify its expression pattern and to determnine its mRNA length (FIG. 1). The murine pre-candidate hybridized to undifferentiated and differentiated ES cells, and to STO cells. In addition, the expression pattern of the murine pre-candidate was analyzed, by RT-PCR using specific primers, in several model systems (FIG. 2):

1. ES cell differentiation: undifferentiated ES cells and 1, 4, 12,19 and 21 days after induction of differentiation of ES cells;

2. mouse embryonic development: 9.5, 11.5, 13.5, 15.5 and 17.5 day-old embryos;

3. ES cell-driven teratocarcinoma formation: 16, 32 and 45 days after ES cell injection;

4. adult mouse tissues: as specified in the detailed description;

5. STO cells: Since STO cells are used as feeder cells during growth of undifferentiated ES cells, it was important to show the specific expression pattern of the pre-candidate in these cells.

MUS_{—}76A11

Mouse gene (Accession: AF110764); also known as RS21-C6 (Tdrg-TL1)

Human Gene (Accession: AF212242); also known as CDA03

Experimental data for mouse gene: Northern hybridization results show one band of 1 Kb in size (FIG. 1). Using RT-PCR analysis, down-regulation of expression was obtained from day 12 of ES cell differentiation. During embryonic development, the expression was down-regulated at day 17.5. No change was detected in the expression level during teratocarcinoma formation. Expression was found in adult mouse liver, pancreas, gut, ovary, fat, lung, spleen, testis, heart and kidney. Low expression was found in brain, placenta and skeletal muscle.

Amino acid homology between the mouse MUS_{—}76A11 predicted gene product and that of the human homolog CDA03 (SEQ ID NO:2) is approximately 80%.

In situ Analysis

Mouse Tissue Multiblock

Antisense probe gave a hybridization signal in the following structures: The most prominent 76A11 signal was observed in testis. The signal localized to the suprabasal layer of seminiferous epithelium in part of tubular profiles. This uneven pattern of expression suggests transcriptional activation of this gene at some intermediate stage of spermatogenesis.

Epithelial lining of the digestive tract represented the other site of prominent 76A11 expression. The hybridization signal was associated with cells located in intestinal crypts and glands of stomach, i.e., with cambial cells responsible for the constant renewal of the epithelial lining.

Weak 76A11 signal (slightly above background) was observed in lymphoid organs: thymus and spleen. In thymus this signal localized to medullar lymphocytes and in spleen, to nodular germinal centers.

The sense probe gave no signal in tissue multiblock.

Embryonic Expression

Embryonic expression of mouse 76A11 was studied on sections of 11.5 and 12.5 days post-conception (dpc) mouse embryos and on whole mounts of 9.5 dpc embryos. The antisense probe gave hybridization signal widely spread throughout embryonic body sections suggesting expression of this gene in multiple cell lineages. Nevertheless, the level of expression varied even within the same structure (e.g., neural tube or heart). The varying levels of expression point to the possible regulation of this gene during differentiation of certain cell types.

Expression in Human Tumors

The in situ hybridization method was employed for the assessment of the expression pattern of the 76A11 human gene in tumor tissue. An EST clone (Accession Number: BG179702) was used as the template for the preparation of the probe specific to the MUS_{—}76A11 gene mRNA. The ³⁵S-labeled antisense riboprobe was synthesized and hybridized to paraffin sections according to standard QBI protocol. Paraffin sections used for the hybridization represented specimens of small cell lung carcinoma (8 cases), breast adenocarcinoma (7 cases) and colorectal adenocarcinoma (8 cases).

Results of in situ hybridization demonstrated weak but detectable expression of the human homolog to the MUS_{—}76A11 gene in 7 out of 8 samples of small cell lung carcinoma, 6 out of 7 samples of breast adenocarcinoma and 6 out of 8 samples of colorectal adenocarcinoma. Significantly, hybridization signal was not detected in normal tissue elements present in sections. These results suggest over-expression of the 76A11 human gene in three types of cancers compared with the normal tissue counterpart.

Sub-cellular Localization: Immunofluorescence Assays

HeLa cells were transiently transfected with a vector expressing MUS_{—}76A11 gene product, tagged with Flag. Forty-eight hours after transfection the cells were fixed with methanol (FIG. 2A,B) or with 3.7% paraformaldehyde (FIG. 2C), and incubated with mouse anti-Flag monoclonal antibody. Cells were then washed and incubated with rhodamine-labeled goat anti-mouse secondary antibody to allow for immunofluorescence detection. After washing, the cells were viewed under a fluorescence microscope.

The 76A11 polypeptide appeared to be localized to the cell membrane (FIG. 2)

### EXAMPLE 2

### Identification and Cloning of Human Homologs of Mouse ESC Candidates

The two basic assumptions underlying and guiding the process of identification of human homologs of mouse genes were:

1. The average percentage of homology between mouse and human genes is 80%, and

2. The highest degree of homology between the two species within the sequence of a given cDNA resides in the amino acid coding region, otherwise designated the open reading frame (ORF).

Accordingly, primers corresponding to regions of the ORF sequence of the desired human gene were designed. These primers were used for screening and detection of human tissue expressing the gene by RT-PCR. Towards this goal total RNA was extracted from the following human cell lines: U266B1 (ATCC#-TIB-196), NCI-H1299 (ATCC#-CRL-5803), K562 (CC1-243) and NAMALWA (ATCC#-CRL-1432). Extraction of RNA was performed using EZ-RNA Isolation Kit (Biological Industries, Beit Haemek, Israel) and mRNA was purified with mRNA Separator Kit (Clontech, Palo Alto, Calif., USA). In addition, mRNAs derived from normal human tissue were purchased from Clontech (Palo Alto, Calif., USA): placenta, whole brain, lung, fetal lung and liver. Oligo (dT)₂₀-primed first-strand cDNA was synthesized from 1 μg each of the different mRNAs, using Thermoscript RT-PCR system (GibcoBRL, Life Technologies, Bethesda, Md., USA). Each of the first-strand cDNAs served as a template for each of the mouse gene-specific primer pairs in a PCR reaction with Expand High Fidelity PCR System (Roche Molecular Biochemicals, Mannheim, Germany). PCR conditions were designed to allow for annealing of mouse-derived primers on the non-identical human template: 92° C.−2 minutes; 5 cycles of 92° C.−30 seconds, 50° C.−30 seconds and 68° C.−1 minute; then 30 cycles of 92° C.−30 seconds, 60° C.−30 seconds and 68° C.−1 minute.

Nucleotide sequences of the resulting human DNA fragments were compared to the original mouse gene sequence. The homologous DNA fragments served as probes for the screening of human full-length cDNA phage libraries or provided initial sequence information for the initiation of 5′ and 3′ RACE reactions using the Marathon cDNA Amplification Kit (Clontech, Palo Alto, Calif., USA). Phage and Marathon cDNA libraries were prepared from the mRNAs on which the positive DNA fragments were synthesized. Synthesis of a phage cDNA library was performed using the SMART cDNA Library Construction Kit (Clontech, Palo Alto, Calif., USA).

In parallel, human EST clones harboring DNA fragments of the desired candidate genes (EST BG179702 for human 76A11) were ordered. Primers corresponding to regions of the ORF sequence of the desired human gene were designed. These primers were used for amplification and cloning of the ORF of the human gene by PCR.

### EXAMPLE 3

### Screening Methods

The gene identified herein can be used as a candidate gene in a screening assay for identifying and isolating compounds which inhibit or stimulate gene transcription or translation or protein expression or activity. The compounds to be screened comprise inter alia small chemical molecules, antibodies or fragments thereof including single chain antibodies, antisense oligonucleotides, antisense DNA or RNA molecules, proteins, polypeptides and peptides including peptido-mimetics and dominant negatives, and expression vectors.

Many types of screening assays are known to those of ordinary skill in the art. The specific assay that is chosen depends to a great extent on the activity of the candidate gene or the protein expressed thereby. Thus, if it is known that the expression product of a candidate gene has enzymatic activity, then an assay which is based on inhibition (or stimulation) of the enzymatic activity can be used. If the candidate protein is known to bind to a ligand or other interactor (interacting molecule), then the assay can be based on the inhibition of such binding or interaction. When the candidate gene is a known gene, then many of its properties can also be known, and these can be used to determine the best screening assay. If the candidate gene is novel, then some analysis and/or experimentation is appropriate in order to determine the best assay to be used to find inhibitors of the activity of that candidate gene. The analysis can involve a sequence analysis to find domains in the sequence that shed light on its activity. Other experimentation described herein to identify the candidate gene and its activity can also be engaged in, so as to identify the type of screen that is appropriate in order to find inhibitors or stimulators (enhancers), as the case may be, for the candidate gene or the protein encoded thereby.

As is well known in the art, the screening assays can be cell-based or non-cell-based. The cell-based assay is performed using eukaryotic cells, and such cell-based systems are particularly relevant in order to measure directly the activity of candidate genes that are involved in proliferation or differentiation. One way of running such a cell-based assay uses tetracycline-inducible (Tet-inducible) gene expression. Tet-inducible gene expression is well known in the art (for example, Hofmann et al., 1996, Proc Natl Acad Sci, 93:5185-5190).

Tet-inducible retrovirus have been designed to incorporate the Self-inactivating (SIN) feature of a 3′LTR enhancer/promoter retroviral deletion mutant. Expression of this vector in cells is virtually undetectable in the presence of tetracycline or other active analogs. However, in the absence of Tet, expression is turned on to maximum within 48 hours after induction, with uniform increased expression of the entire population of cells that harbor the inducible retrovirus, thus indicating that expression is regulated uniformly within the infected cell population.

When dealing with candidate genes having e.g., proliferation function such as gene 76A11, Tet-inducible expression increases proliferation in target cells, i.e., reduces the doubling time. One can screen for chemical compounds able to rescue the cells from the gene-triggered increase in proliferation. When dealing with candidate genes having anti-proliferation function, Tet-inducible expression reduces proliferation in target cells. One can screen for chemical compounds able to rescue the cells from the gene-triggered reduction in proliferation.

If the gene product of the candidate gene phosphorylates a specific target polypeptide, a specific reporter gene construct can be designed such that phosphorylation of this reporter gene product causes its activation, which can be followed by a color reaction. The candidate gene can be specifically induced, using the Tet-inducible system discussed above, and a comparison of induced versus non-induced genes provides a measure of reporter gene activation.

In a similar indirect assay, a reporter system can be designed that responds to changes in protein-protein interaction of the candidate polypeptide. If the reporter responds to actual interaction with the candidate protein, a color reaction occurs.

One can also measure inhibition or stimulation of reporter gene activity by modulation of its expression levels via the specific candidate promoter or other regulatory elements. A specific promoter or regulatory element controlling the activity of a candidate gene is defined by methods well known in the art. A reporter gene is constructed that is controlled by the specific candidate gene promoter or regulatory elements. The DNA containing the specific promoter or regulatory agent is actually linked to the gene encoding the reporter. Reporter activity depends upon specific activation of the promoter or regulatory element. Thus, inhibition or stimulation of the reporter gene is a direct assay of inhibition or stimulation of the candidate gene, respectively (e.g., Komarov et al., 1999, Science, 285:1733-1737; Storz et al., 1999, Analytical Biochemistry, 276:97-104).

Design of various non-cell-based screening assays are also well within the skill of those of ordinary skill in the art. For example, if enzymatic activity is to be measured, such as if the candidate protein has a kinase activity, the target protein can be defined and specific phosphorylation of the target can be followed. The assay can involve either inhibition of target phosphorylation or stimulation of target phosphorylation, both types of assay being well known in the art, (e.g., Mohney et al., 1998 J.Neuroscience, 18:5285; Tang et al., 1997, J Clin. Invest. 100:1180) for measurement of kinase activity.

It will be appreciated that, based on knowledge of the 76A11 polypeptide, it is possible to devise a non cell-based assay for screening for, i.e. identifying compounds that modulate proliferation or differentiation through the human 76A11 polypeptide. The proliferation or differentiation effect of the 76A11 polypeptide may be due to the specific binding or interaction of part or all of the 76A11 polypeptide to a different species such as, without limitation, a factor, molecule, or specific binding substance, and this effect may be monitored by linking this specific binding or interaction to a signaling system. We thus wish to identify compounds which, for example, modulate or disturb this specific interaction of the 76A11 polypeptide with such species.

Therefore, in a non-cell-based embodiment there is provided a method for identifying a test chemical compound that modulates proliferation or differentiation of cells that comprises the steps of:

a. providing a mixture comprising polypeptide 76A11,

b. contacting said mixture with a test substance under conditions which normally lead to 76A11 activity, and

c. determining whether the activity of said polypeptide or fragment is affected by said compound.

One can also measure in vitro interaction of a candidate polypeptide with interactors. In this screen, the candidate polypeptide is immobilized on beads. An interactor, such as a receptor ligand, is radioactively labeled and added. When it binds to the candidate polypeptide on the bead, the amount of radioactivity carried on the beads (due to interaction with the candidate polypeptide) can be measured. The assay indicates inhibition of the interaction by measuring the amount of radioactivity on the bead.

Any of the screening assays according to the present invention can include a step of identifying the chemical compound (as described above) which tests positive in the assay, and can also include the further step of producing as a medicament that which has been so identified. It can also include steps of improving the chemical compound to increase its desired activity before incorporating the improved chemical compound into a medicament. It is considered that medicaments comprising such compounds are part of the present invention. The use of any such compounds identified for inhibition or stimulation of proliferation or differentiation is also considered to be part of the present invention.

### EXAMPLE 4

### Growth Rate Analysis of Differentiated Embryonic Stem Cells

MUS_{—}76A11 cDNA was over-expressed in embryonic stem cells. Clones of such over-expressing cells were induced to differentiate by generation of EBs, as described above. The EBs were then plated out in 24-well plates. In comparison with control EBs that did not over-express MUS_{—}76A11, the over-expressing clones were clearly larger according to visual inspection, as early as by day 6. This result suggests an effect of MUS_{—}76A11 over-expression on the division rate of cells and supports the possible role of 76A11 in stem cell expansion.

### EXAMPLE 5

### Growth Rate Analysis of Undifferentiated Embryonic Stem Cells

Stable clones of both undifferentiated ES cells that over-express MUS_{—}76A11 and non-expressing clones are plated in 96-well tissue culture plates and grown for 1, 2 or 3 days. The cells are then subjected to the XTT assay, performed as per manufacturer's instructions, in order to compare the cell number of each over-expressing and non-expressing clone. The XTT assay (Biological Industries, Bet Ha'emek, ISRAEL (Cat. No. 203001000)) is a color-based assay, wherein the intensity of the color reaction (the result of enzymatic activity) correlates directly with cell number.

### EXAMPLE 6

### Stem Cell Expansion

The polypeptide encoded by the human homolog of MUS_{—}76A11 viz., gene 76A11, is contacted by e.g. addition to the medium with a stem cell population. The mixture is incubated and the number of stem cells in the resultant stem cell population is determined, as compared to a control e.g. where denatured polypeptide is added. Increase in cell population of the treated cells compared to the control indicates that the 76A11polypeptide may promote stem cell expansion.

### EXAMPLE 7

### Manufacture and use of the Claimed Products

The claimed polynucleotide of the subject invention can be constructed by using a commercially available DNA synthesizing machine and SEQ ID NO:1. For example, overlapping pairs of chemically synthesized fragments of the gene can be ligated using methods well known in the art (e.g., see U.S. Pat. No. 6,121,426).

Another means of isolating a claimed polynucleotide, e.g., the polynucleotide of SEQ ID NO:1, is to obtain a natural or artificially designed DNA fragment based on that sequence. This DNA fragment is labeled by means of suitable labeling systems which are well known to those of skill in the art; see, e.g., Davis et al. (1986). The fragment is then used as a probe to screen a lambda phage cDNA library or a plasmid cDNA library using methods well known in the art; see, generally, Sambrook et al., Molecular Cloning: A Laboratory Manual, Cold Spring Harbor Laboratory Press, New York (1989), in Ausubel et al., Current Protocols in Molecular Biology, John Wiley and Sons, Baltimore, Md. (1989),

Colonies can be identified which contain clones related to the cDNA probe and these clones can be purified by known methods. The ends of the newly purified clones are then sequenced to identify full-length sequences. Complete sequencing of full-length clones is performed by enzymatic digestion or primer walking. A similar screening and clone selection approach can be applied to clones from a genomic DNA library. The entire naturally occurring cDNA or gene sequence, including any allelic variations thereof, will all have the same utility as discussed above for the identified polynucleotide.

The claimed polynucleotide can be used inter alia as a probe for diagnostic work. It can be used to diagnose cancerous cells, whereby the gene of nucleotide sequence as presented in SEQ ID NO:1 is over-expressed and there are, thus, high levels of mRNA gene transcripts.

The claimed polypeptide can be produced by making a synthetic polypeptide, using a commercially available machine and SEQ ID NO:2. Another means of making the claimed polypeptide is to clone cDNA or a fragment thereof, using SEQ ID NO:1, and express the resulting polypeptide, using methods known in the art.

A preferred method of producing the 76A11 polypeptide is to clone a polynucleotide comprising the protein-encoding sequence of the 76A11 gene into an expression vector and culture the cell harboring the vector so as to express the encoded polypeptide, and then purify the resulting, polypeptide, all performed using methods known in the art as described in, for example, Marshak et al., 1996, Strategies for Protein Purification and Characterization. A laboratory course manual. CSHL Press. In addition, see Bibl Haematol., 1965, 23:1165-1174; Appl Microbiol., 1967, July;15(4):851-856; Can J Biochem. 1968 May;46(5):441-444; Biochemistry, 1968, July;7(7):2574-2580; Arch Biochem Biophys., 1968, September 10;126(3):746-772; Biochem Biophys Res Commun., 1970, February 20;38(4):825-830.

The expression vector can include a promoter for controlling transcription of the heterologous material and can be either a constitutive or inducible promoter to allow selective transcription. Enhancers that can be required to obtain necessary transcription levels can optionally be included. The expression vehicle can also include a selection gene.

Additionally the naturally occurring polypeptide can be isolated and purified from an organism or from cultured cells using methods known in the art.

The claimed polypeptide can be used for expansion of cells as described in detail above. Additionally, the claimed polypeptide can be used for the manufacture of antibodies, which can be used to diagnose cancerous cells, whereby the gene of SEQ ID NO:1 is over-expressed and there are, thus, high levels of the polypeptide of SEQ ID NO:2. Furthermore, it is well known that proteins and polypeptides are nutritious and edible.

Throughout this application various publications, including United States patents, are referenced by author and year, and patents by number. The disclosures of these publications and patents in their entireties are hereby incorporated by reference into this application in order to describe more fully the state of the art to which this invention pertains.

The invention has been described in an illustrative manner, and it is to be understood that the terminology that has been used is intended to be in the nature of words of description rather than of limitation.

Obviously, many modifications and variations of the present invention are possible in light of the above teachings. It is, therefore, to be understood that within the scope of the described invention, the invention may be practiced otherwise than as specifically described.

What is claimed is:

1. A method for treating a subject suffering from a disease, wherein the disease is characterized by aberrant cellular proliferation or aberrant cellular differentiation, comprising administering to the subject an antagonist of a polypeptide, wherein the polypeptide has an amino acid sequence as presented in SEQ ID NO:2.

2. The method of claim 1, wherein the polypeptide is administered in conjunction with a chemotherapeutic agent.

3. The method of claim 1, wherein the antagonist is an antibody.

4. The method of claim 1, wherein the antagonist is a small molecule.

5. The method of claim 1, wherein the disease is a cancer.

6. The method of claim 5, wherein the cancer is selected from the group consisting of leukemia, lymphoma, myeloma and carcinoma.

7. The method of claim 6, wherein the carcinoma is selected from the group consisting of small cell lung carcinoma and adenocarcinoma.

8. The method of claim 7, wherein the adenocarcinoma is selected from a group consisting of breast adenocarcinoma and colorectal adenocarcinoma.

9. Use of a polynucleotide as a probe for identifying undifferentiated embryonic stem cells, wherein the polynucleotide comprises a nucleotide sequence as presented in SEQ ID NO:1.

10. Use of the 76A11 polypeptide in vivo or ex vivo for expansion of adult somatic stem cells.

11. The use of claim 10, wherein the polypeptide has a sequence as presented in SEQ ID NO:2.

12. The use of claim 10, whereby the stem cells are selected from a group consisting of hematopoietic, neuronal, liver, pancreatic beta-, muscle, mesenchymal, skin stem cells and osteoprogenitor cells.

13. The use of claim 10, wherein the expansion of adult somatic stem cells comprises:
a. isolating a population of adult somatic stem cells,
b. contacting the cells with a preparation comprising a polypeptide of SEQ ID NO:2,
c. incubating the preparation with the cells under conditions permitting expansion of the cells, and
d. harvesting the expanded stem cell population.

14. Use of an antibody directed to an epitope presented by the polypeptide comprising the amino acid sequence as presented in SEQ ID NO:2 for detection of cancerous cells or of undifferentiated stem cells.

15. Use of a polynucleotide comprising the nucleotide sequence as presented in SEQ ID NO:1 or fragments thereof for identifying cancerous cells.

16. A differentiation factor encoded by the DNA sequence as presented in SEQ ID NO:1.

17. A proliferation factor encoded by the DNA sequence as presented in SEQ ID NO:1.

18. A method for identifying a chemical compound that modulates proliferation or differentiation of cells which comprises:
a. contacting a cell expressing a gene comprising the sequence as presented in SEQ ID NO:1 with the chemical compound, under conditions permitting expression of the gene; and
b. determining whether the proliferation or differentiation of the cell is stimulated or inhibited in the presence of the compound, as compared to a control.

19. The method according to claim 18, wherein said cell in said contacting step has been transfected by the gene being expressed.

20. A method for identifying a test chemical compound that modulates proliferation or differentiation of cells which comprises the steps of:
a. providing a mixture comprising polypeptide 76A11,
b. contacting said mixture with a test substance under conditions which normally lead to 76A11 activity, and
c. determining whether the activity of said polypeptide is affected by said compound.

21. A method of preparing a pharmaceutical composition which comprises determining whether a chemical compound stimulates or inhibits a proliferation or differentiation gene by:
using the method of claim 18, and admixing said compound with a pharmaceutically acceptable carrier.

22. A method of preparing a pharmaceutical composition which comprises determining whether a chemical compound stimulates or inhibits a proliferation or differentiation gene by:
using the method of claim 20, and admixing said compound with a pharmaceutically acceptable carrier.

23. The method of claim 21, wherein the pharmaceutical composition additionally comprises a chemotherapeutic agent.

24. The method of claim 22, wherein the pharmaceutical composition additionally comprises a chemotherapeutic agent.
